(19) United States
(12) Patent Application Publication
(10) Pub. No.: US 2025/0018346 A1
(43) Pub. Date: Jan. 16, 2025

(54) SELF-PRESERVING BIODEGRADABLE ENVIRONMENTAL DNA FILTER

(71) Applicant: Smith-Root, Inc., Vancouver, WA (US)

(72) Inventors: Austen Clouse Thomas, Port Ludlow, WA (US); Jesse Andrew Howard, Vancouver, WA (US)

(21) Appl. No.: 18/904,010

(22) Filed: Oct. 1, 2024

Related U.S. Application Data
(63) Continuation-in-part of application No. 17/967,550, filed on Oct. 17, 2022, now Pat. No. 12,145,107.
Application No. 17/967,550 is a continuation of application No. 16/780,816, filed on Feb. 3, 2020, now Pat. No. 11,471,837.
(60) Provisional application No. 62/800,248, filed on Feb. 1, 2019.

(51) Int. Cl. B01D 67/00; B01D 63/08; B01D 69/06; C12N 15/10; C12Q 1/6806; G01N 1/10; G01N 1/14; G01N 1/40

(52) U.S. Cl. CPC B01D 67/0088; B01D 63/087; B01D 69/06; C12N 15/1017; C12Q 1/6806; G01N 1/14; G01N 1/4005; B01D 2315/08; B01D 2325/36; G01N 2001/1025; G01N 2001/4088

(57) ABSTRACT

A filter assembly includes a housing having an inlet port, an outlet port, and an interior compartment, the inlet port and the outlet port fluidically coupled to form a flow path in the interior compartment, a filter membrane in the flow path, the filter membrane having a porosity capable of capturing biological molecules as liquid flows along the flow path, a fluid screen in the flow path on an opposite side of the filter membrane from the inlet port, and a desiccating element in the flow path comprised of one of a hygroscopic or hydrophilic material selected to absorb moisture from the filter membrane at ambient temperature.

### CROSS REFERENCE TO RELATED APPLICATIONS

This application is a continuation-in-part of U.S. Non-provisional patent application Ser. No. 17/967,550, filed Oct. 17, 2022, which is a continuation of U.S. Non-provisional patent application Ser. No. 16/780,816 filed Feb. 3, 2020, now U.S. Pat. No. 11,471,837, which claims priority from U.S. Provisional Patent Application No. 62/800,248, filed 1 Feb. 2019, all of which are incorporated herein by reference in its entirety.

### TECHNICAL FIELD

This disclosure relates to an apparatus, systems and methods for the filtering and preserving of environmental DNA.

### BACKGROUND

Field sampled environmental DNA from lakes, streams, and oceans is used to both identify the species that are present in the body of water and try to quantify the population of individual species. The technique of analyzing field sampled environmental DNA involves the field collection of aqueous samples from water bodies (streams, lakes, swamps, effluent discharges, etc.) and then testing those samples using DNA replication protocols.

DNA samples are subject to degradation when they are extracted for sampling. This degradation can be due to enzyme activity and/or chemicals that are added to the sample. During sampling, it is important to prepare and preserve samples in the field easily, quickly, and efficiently, so samples that are analyzed for environmental DNA are viable.

Some of the current protocols for the collection of field sampled environmental DNA involve the preparation and assembly of a sampling apparatus from separate components. One of the current drawbacks of field sampling methods is that the sampling of DNA using environmental DNA filters must be preserved to maintain the viability of the DNA samples. Preservation of the filters can involve transferring the filters to a chemical preservative, be desiccated, or require cold storage in the field. In one method the field preservation of DNA involves the field sampling technician opening up the filter cartridge; folding the environmental DNA filter with a pair of sterile forceps; then inserting of the filter into a vial or bag containing DNA preservative. These transfer steps can be challenging to perform by a field technician and there is an increased risk of sample contamination by inadvertent DNA contamination. Some field sampled environmental DNA technicians use fully encapsulated filters and then place the full filter cartridge in cold storage to preserve the DNA samples. However, transport of cold storage materials into the field may be costly or logistically prohibitive.

As the number of practitioners using environmental DNA survey methods has increased rapidly in recent years, the standards for what is considered acceptable environmental DNA practice have also increased. More emphasis is being placed on a rigorous set of lab and field protocols that minimize the potential for DNA contamination from myriad potential sources. New tools are therefore needed to help environmental DNA practitioners achieve these high standards both efficiently and cost-effectively. There is an indication that self-preserving environmental DNA filter cartridges are a viable alternative to standard environmental DNA preservation methods that help to reduce the risk of sample contamination, minimize protocol steps, and result in less plastic waste.

Therefore, there is a need to improve the preservation of field environmental DNA samples at the point of sampling in the field by the use of a desiccating filter cartridge. While field biologists and molecular biologists are focused on using the sterile technique as the means in which to prevent sample contamination, more and more scientists and researchers and even citizens of the general population who are not trained to collect and prepare DNA are seeking easier methodology for sampling environmental DNA in aquatic environments without causing contamination.

### BRIEF DESCRIPTION OF THE DRAWINGS

FIG. 1 is the sampling system for the collection of environmental DNA.

FIG. 2 is a close-up view of the sampling pole that holds the filter assembly cartridge.

FIG. 3 is an exploded side view of the filter assembly cartridge.

FIGS. 4A, 4B, and 4C are views of the top of the filter assembly cartridge.

FIGS. 5A, 5B, and 5C are views of the desiccating bottom of the filter assembly cartridge.

FIG. 6 depicts comparative test results.

FIG. 7 shows a method for using the filter assembly cartridge.

FIG. 8 shows a pictorial diagram of some of the steps in using the filter assembly cartridge.

FIG. 9A is a cross-sectional view of an embodiment of the filter assembly cartridge of FIG. 8, in accordance with implementations of the present disclosure.

FIG. 9B is a close-up view of an embodiment of the filter assembly cartridge of FIG. 9, in accordance with implementations of the present disclosure.

FIG. 9C is a cross-sectional view of an embodiment of the filter assembly cartridge of FIG. 9, having the filter removed to view the desiccant, in accordance with implementations of the present disclosure.

FIG. 10 shows an alternative embodiment of a filter assembly cartridge.

FIG. 11 shows an alternative embodiment of a filter assembly cartridge.

FIG. 12 shows an exploded view of an alternative embodiment of a filter assembly cartridge.

FIG. 13 shows an exploded view of an alternative embodiment of a filter assembly cartridge.

FIG. 14 shows an exploded view of an alternative embodiment of a filter assembly cartridge.

### DETAILED DESCRIPTION

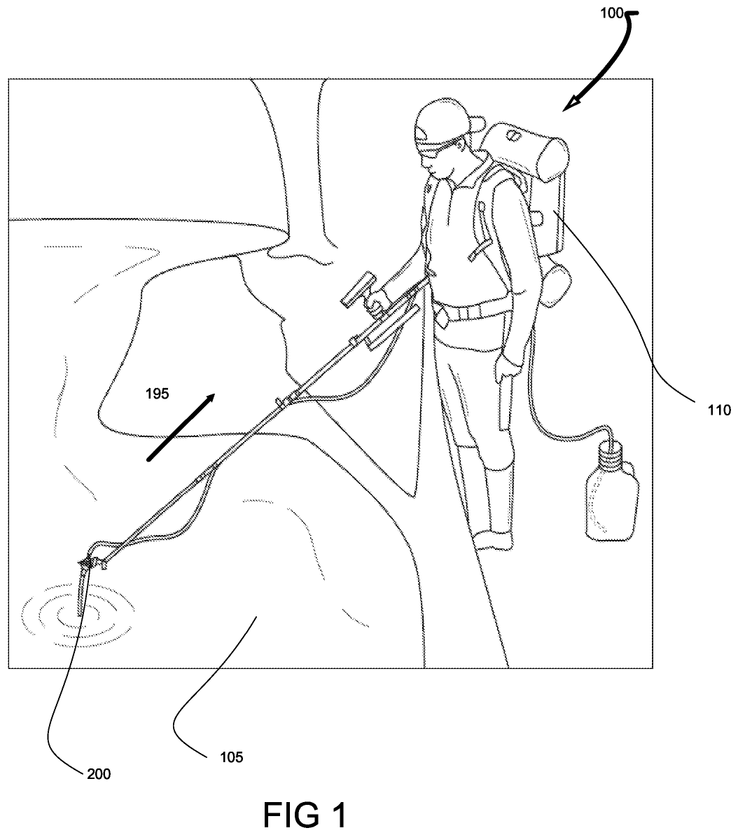

Now referring to FIG. 1 which illustrates the environmental DNA collection system 100. The sampling backpack 110 is used to draw water 105 through a filter cartridge assembly 200. The flow of the water 195, is from the body of water, through for this example, the Smith-Root eDNA backpack sampling system is depicted in this application, but alternate configurations where the sampling system is not configured as a backpack would be suitable, such as the sampling system could be mounted in a mobile case, a cart, or located inside a laboratory building. In this particular implementation, the water containing the environmental DNA is drawn upwards through the filter cartridge assembly 200 and then to the sampling backpack 110.

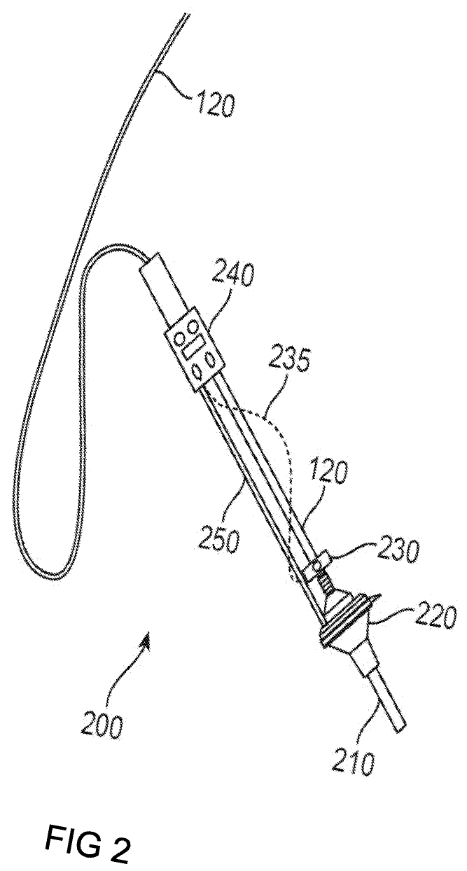

Now referring to FIG. 2 which is the probe assembly which holds the filter cartridge assembly 220. The probe assembly aids in sampling by a placing the filter cartridge assembly 220 close to the body of water. Now referring to FIG. 2 which depicts the inventive subject matter of the water collection and filtration module 200, consisting of the induction tube 210, the filter cartridge assembly 220 and the pump tubing 120. In some embodiments, the flow rate sensor 230 may be located inside the casing for power and control module 100. The induction tube 210 draws water from the sampling source, using the suction generated by the pump, and directs the water into the filter cartridge assembly 220. Water flows through the filter cartridge assembly 220, where particulate is captured, and then flows into the pump tubing 120.

In the preferred embodiment, the amount of water drawn through the filter cartridge assembly 220 has a minimum flow rate of 0.1 L/min to a maximum flow rate of 1.4 L/min. The accuracy of fluid volume measurement is approximately greater than 90% at a rate of 0.1 L/min. The preferred orifice size is approximately 0.25 inner diameter tubing.

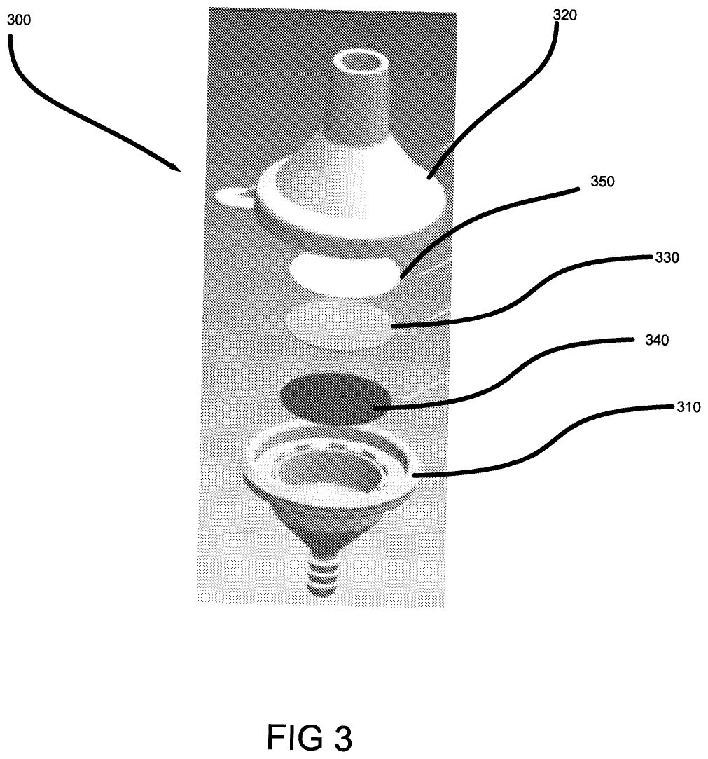

Now referring to FIG. 3 which has an exploded view of the filter cartridge assembly 220 having a top 320, a filter membrane 350, a filter backer 330, a steel mesh 340, and a desiccating bottom 310. The water flows 195 through the top 320, through the filter membrane 350, the filter backer 330, the steel mesh 340, and out the desiccating bottom 310.

The filter membrane 350 should have a pore size that provides for the capture of environmental DNA but allows for the free passage of the liquid part of the sample. The filter membrane for sampling environmental DNA should range from 0.22 μm (microns) to 40.0 μm (microns).

The desiccating bottom 310 will be a hydrophilic material that is active at a range of field and laboratory temperatures (1-43 degrees Celsius) and capable of rapidly absorbing moisture from the filter membrane 350 encased in the filter cartridge. The filter membrane 350 materials can be Cellulose Acetate, Glass Fiber, MCE (Mixed Cellulose Ester), Nitrocellulose, Nylon, PCTE (Polycarbonate track etch), PES (Polyethersulfone), PETE (Polyester track etch), PAN (Polyacrylonitrile), PEEK (Polyether Ether Ketone), PVDF (Polyvinylidene fluoride)

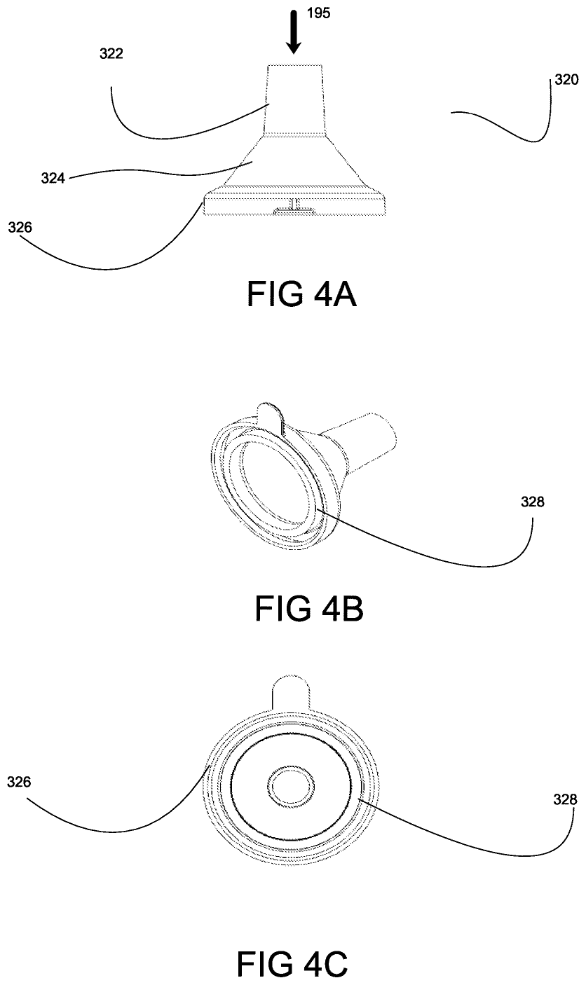

Now referring to FIGS. 4A, 4B, and 4C illustrating various orientations of the top 320. The top 320 has a tubular section 322 that allows the passage of liquids with an inlet port, the tubular section 322 is connected to a flared conical section 324, that terminates in a flexible lip structure 326. An embodiment of the top 320 closely resembles a funnel-like structure, but any corresponding structure is permissible, including, but not limited to cylindrical configurations, rectangular, cubicle, or other configurations. The flexible lip structure 326 with the flexible lip structure 326 has an inner mating ring 328 allows for a compression fit to the desiccating bottom 310. The top 320 may be made of any substance that is inert and does not contaminate the flow of water 195 or degrade DNA. While a flexible lip structure 326 is preferred, other coupling configurations that provide a seal are permissible, such as, inflexible compression fit, threaded couplings, or other similar couplings that allows passage of water without leakage.

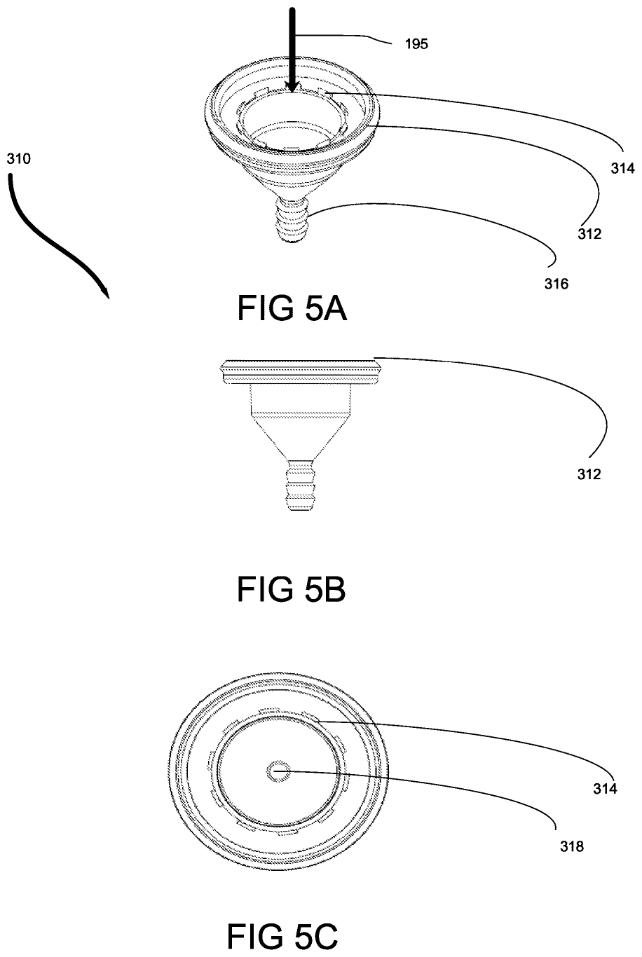

Now referring to FIGS. 5A, 5B, and 5C illustrating various orientations of the desiccating bottom 310. The desiccating bottom 310 has a tubular section 316 that allows the passage of liquids with an out of the outlet port, the tubular section 316 is connected to a flared conical section, which terminates in a ridged lip structure 312. An embodiment of the desiccating bottom 310 closely resembles a funnel-like structure, but any corresponding structure is permissible, including, but not limited to cylindrical configurations, rectangular, cubicle, or other configurations. The ridged lip structure 312 allows for a compression fit to the top 320. The top 320 may be made of any substance that is inert and does not contaminate the flow of water 195 or degrade DNA. While a ridged lip structure 312 is preferred, other coupling configurations that provide a seal are permissible, such as, inflexible compression fit, threaded couplings, or other similar couplings that all passage of water without leakage.

Now with reference to FIG. 3 and further in reference to FIG. 4 and FIG. 5, during operation, the filter cartridge assembly 220 draws water through the induction tube 210, the filter membrane 350, filter backer 330, steel mesh 340, and then to the pump tubing 210. Environmental DNA is captured on the filter membrane 350. The bottom 310 of the filter cartridge assembly 220 is made of a hydrophilic substance that absorbs water. This absorption of water assists in the desiccation of the filter membrane after sampling and preserves the membrane-captured fragments of eDNA for subsequent amplification via the polymerase chain reaction (PCR).

The filter membrane 350 may be dimensioned to any size, although use of an approximately 47 mm diameter is preferred.

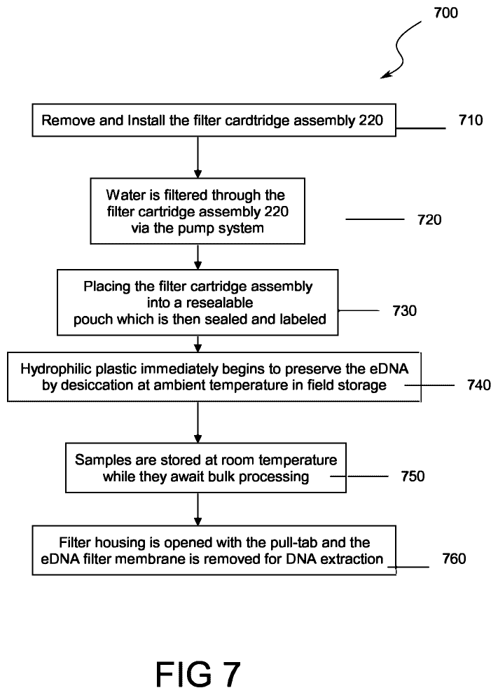

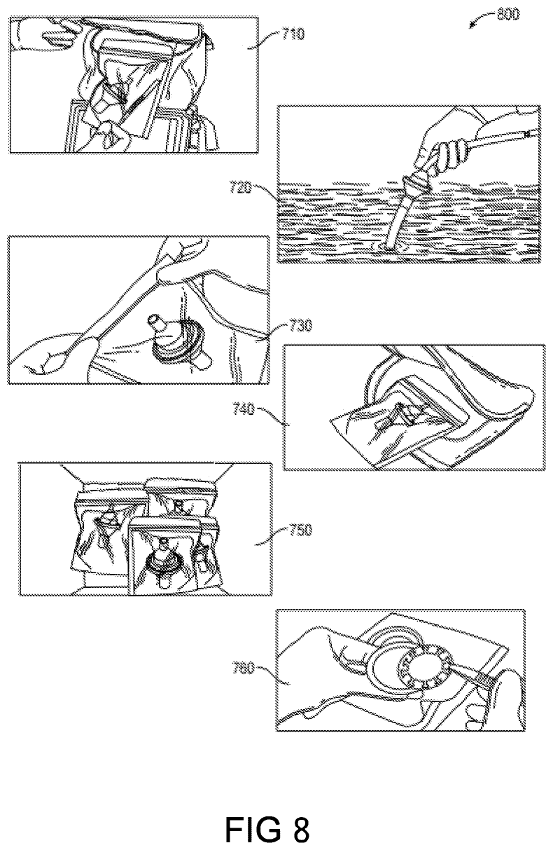

Now referring to FIG. 7 (flowchart) and FIG. 8 (diagrammatic representation), which illustrates the method of installing and using the self-preserving environmental DNA filter cartridge assembly 220. A filter cartridge assembly storage packet is opened 710 containing the filter cartridge assembly 220 and induction tube 210. A water sample is collected from a water body and filtered 720 through the filter cartridge assembly 220 using the attachment to a pump system as illustrated in 200 of FIG. 2.

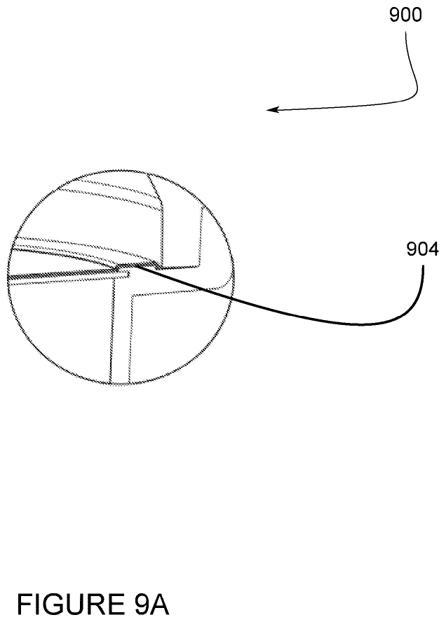

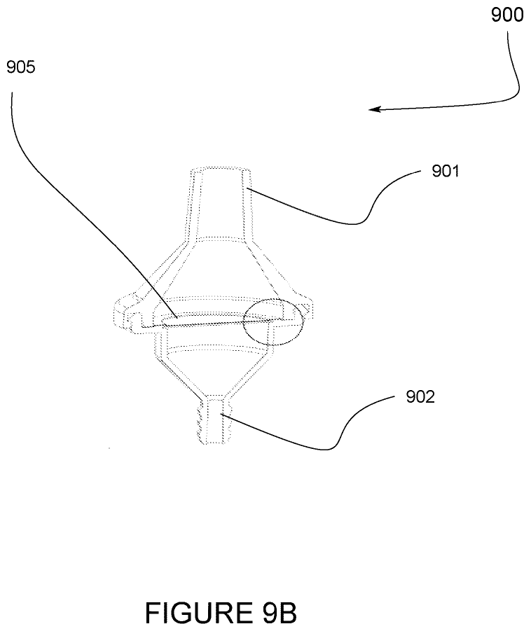

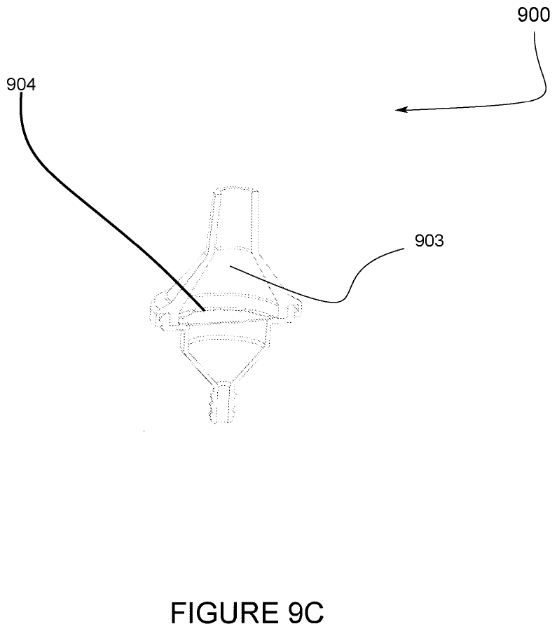

Now referring to FIGS. 9A, 9B, 9C that depict cutaway views of the self-preserving filter housing and internal components, with FIG. 9B illustrating the assembly with a filter membrane in place, FIG. 9A illustrating a detail view, and FIG. 9C illustrating the assembly without the filter membrane in place to reveal the underlying structure. Here the filter enclosure includes an inlet port 901 on one end of the enclosure, an outlet port 902 on the opposite end of the enclosure, and an internal cavity 903 formed by the non-permeable enclosure that encapsulates the internal components. Within the enclosure, there is an internal desiccating element 904 configured to preserve environmental DNA on a filter by desiccation. The desiccating element is illustrated as multiple raised structures that create radially distributed contact points (moisture absorption locations) with the internal filter. Also within the enclosure is a filter 905 having a porosity capable of filtering DNA from a passage of liquid that is mounted proximate to, or in direct contact with, the desiccating element.

The filter cartridge assembly 220 is removed from the pump system placed unopened back into the resealable pouch 730 which is then sealed 730. Upon storage, the hydrophilic plastic immediately begins to preserve the environmental DNA by desiccation at ambient temperature 40. The field cartridge assembly may be stored at room temperature while they await bulk processing 750. The filter cartridge assembly 220 is opened by separating the top 320 from the desiccating bottom 310 and removing the filter membrane 350 for DNA extraction and analysis.

Use of the Filter Cartridge Assembly with Other Sampling Systems

The filter cartridge assembly 220 is not limited to only being used with the field sampling system described above. The filter cartridge assembly 220 may be used with any field sampling apparatus that will draw an environmental sample through a tube. The inlet and outlet ports of the filter cartridge assembly 200 may also be mated to other field sampling systems by using simply tube adapters on either the inlet or outlet ports.

### Methods for the Preservation of the Field Sampled Environmental DNA

The steps for preserving the environmental DNA are shown in flow chart 700 for a self-preserving environmental DNA filter process with reference to the pictorial diagram 800.

A filter packet is opened containing the pre-loaded inline filter cartridge made with hydrophilic bottom 310.

The extension tube (in packet) and suction tubing are attached to the filter cartridge; pump is activated to begin filtration.

The suction tubing is then placed in the body of water (lake, stream, etc).

Water is then filtered through the filter membrane by attachment to a pump system.

The filter cartridge is placed into a resealable pouch which is then sealed and labeled.

The hydrophilic plastic immediately begins to preserve the environmental DNA by desiccation at ambient temperature.

The filter cartridge is opened with the pull-tab and the environmental DNA filter membrane is removed for DNA extraction 260.

Another embodiment may also be employed.

Sample packet containing a filter cartridge assembly is removed from seal packet.

The extension tube (in packet) and suction tubing are attached to filter cartridge; pump is activated to begin filtration.

When “low flow” alarm sounds or target volume is reached, the filter cartridge is inverted and elevated to filter all remaining water in cartridge and clear the suction line.

Seal is cracked (not opened) and the pump continues to run for approximately 20 seconds to air dry the filter membrane.

The extension tube is removed from the filter cartridge and discarded.

The self-preserving filter cartridge is placed back into the original packaging.

The package is resealed, and the filter cartridge material immediately begins preserving the captured DNA via desiccation.

The sample is labeled and placed back inside field storage at ambient temperature.

Once in the laboratory, the technician removes the filter cartridge with preserved filter membrane inside.

The filter cartridge is opened by the pull-tab, revealing the environmental DNA filter membrane.

The environmental DNA filter membrane is removed from the cartridge with sterile forceps for DNA extraction, and the filter backer remains in the cartridge. All elements other than the environmental DNA filter membrane are then discarded.

### Validation and Testing of the Field Sampled Environmental DNA

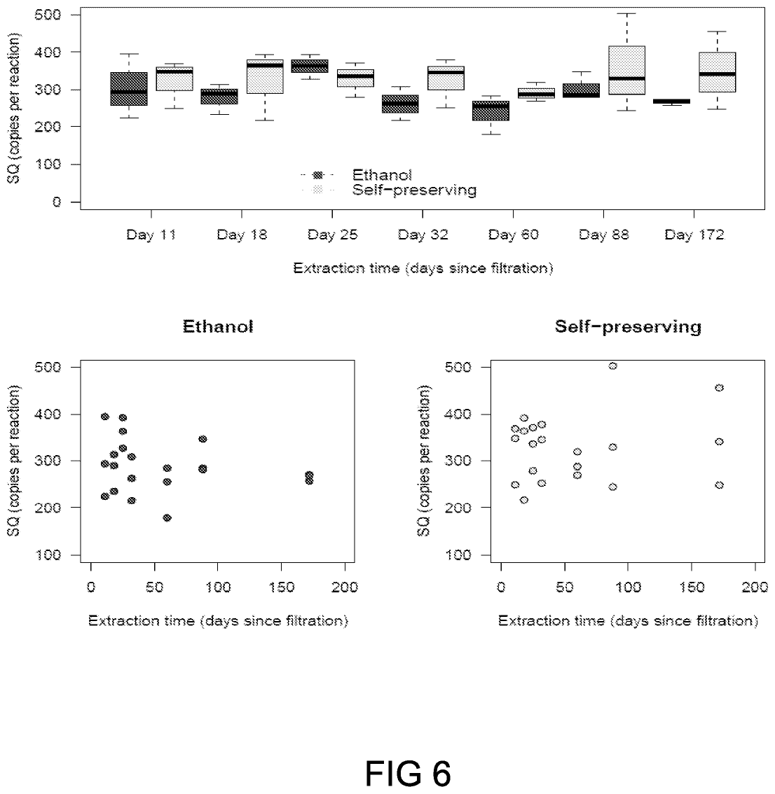

Now referring to FIG. 6, environmental DNA preservation capabilities of the hydrophilic filter cartridges were compared to the standard ethanol field preservation method using a mesocosm experiment. Replicate water samples were collected and filtered from a tank containing a single suspended concentration of New Zealand mudsnail (Potamopyrgus antipodarum) environmental DNA in river water. Half of the samples were preserved in ethanol and the other half were allowed to be self-preserved by placing the filter cartridge back in original packaging. The mudsnail environmental DNA preserved on filters was then extracted at multiple time points and quantified by qPCR to determine the degree of environmental DNA degradation over time for each preservation method.

A description of the materials and methods involved in the Mesocosm setup is as follows: A total of 88 L of environmental water was collected from a local creek and transferred to a 151 L total-volume test tank (91 cm Lx46 cm Wx21 cm H) held in a wet lab. Environmental water from the creek was used to ensure that the experiment accounted for naturally occurring environmental PCR inhibitors. An additional 17 L of water from a rearing tank containing a small population of New Zealand mudsnails CIOO individuals) was added to the test tank to create a total volume of 105 L of water with known New Zealand mudsnail environmental DNA. Water in the test tank was circulated throughout the experiment using a gyre pump (Maxspect XF250-5300 GPH)—this ensured that New Zealand mudsnail environmental DNA was kept suspended and mixed throughout the tank. Detectability of New Zealand mudsnail environmental DNA was confirmed in the test tank prior to replicate sample collection by testing with Biomeme handheld qPCR.

A description of the water filtration and preservation is as follows: Water was filtered from the test tank using the Smith-Root environmental DNA sampler with single-use filter packets. The filter cartridges, contained in packets were pre-loaded with 1.0 μm (47 mm diameter) polyether sulfone (PES) filter membranes, which are 50% comprised of an injection-molded hydrophilic plastic. Filtration parameters on the environmental DNA sampler were standardized for all samples at 1.0 L/min flow rate, 13 psi pressure threshold, and 0.5 L target volume. Environmental DNA Filter samples were collected for both preservation treatments (ethanol, self-preserved) and labeled for DNA extraction at 7 time points post-collection: 11 days, 18 days, 25 days, 32 days, 60 days, 88 days, 172 days. Three replicate filter samples were collected for each combination of preservation method and extraction time point, for a total of 42 samples (21 ethanol, 21 self-preserved).

After filtration, the filter membranes for ethanol preservation were immediately removed from the cartridge, folded, and inserted into individual 2 mL test tubes filled with approximately 1.25 mL of 200 proof reagent-grade ethanol to sufficiently cover the sample. The sampling and preservation procedure for the self-preserved filters were modified to minimize the amount of moisture that the hydrophilic plastic was required to absorb. At the end of a filtration cycle the environmental DNA sampler produces an audible “low-flow” alarm-indicating that all water in the suction tubing has been metered and filtration is complete. For the self-preserved samples in this experiment, the pump was allowed to continue running for 20 seconds at this stage to effectively air dry the filter membrane. After the drying step, the filter cartridge was placed back into the foil pouch and rescaled using the zip-type sealing strip for preservation.

A description of the environmental DNA quantification and analysis is as follows: Samples of both preservation treatments were shipped overnight to the Goldberg lab at Washington State University and stored at room temperature until their prescribed DNA extraction time point. DNA was extracted from filters following the laboratory's standard protocol: filter homogenization via QIAshredder (Qiagen, Inc.), DNA extraction with the DNeasy Blood & Tissue Kit (Qiagen, Inc.) and 100 ul elution. Mudsnail environmental DNA on each filter was detected and quantified by triplicate qPCR using a custom assay previously described in Goldberg et al. (2013): NZMS F-TGTTTCAAGTGTGCTGGTTTAYA, NZMS Probe-6FAMCCTCGACCAATATGTAAAT-MGB, NZMS R-CAAATGGRGCTAGTTGATTCTTT, using PCR reactions with QuantiTect Multiplex PCR Mix (Qiagen, Inc.). Recommended duplexing concentrations were used (0.4 mM of each primer, and 0.2 mM of each probe) on a BioRad CFX96 and downsized to 10-mL reactions. Cycling was 15 min initial denature at 95 C, followed by 50 cycles of 94 C for 60 sand 60 C for 60 s. An exogenous internal positive control (Applied Biosystems) was included in each well as a test for inhibition. Reaction Starting Quantity (SQ) was calculated by a standard curve comprising 10, 100, 1000, and 10000 copies per well of gBlock standard (IDT, Inc.) and run with each plate of filter sample extracts.

Mudsnail environmental DNA degradation over the storage period was compared between the two preservation treatments and the five extraction time points using the SQ values produced by qPCR. First, the SQ values from the three qPCR replicates were average for each filter sample. A two-way ANOVA was then performed on the filter SQ values, using “preservation method” and “extraction time” as predictor variables. The base aov function in R (R Core Team 2014), treating preservation method as a factor with two levels and extraction time (in days) as a continuous integer variable. We tested for the simple main effects and for an interaction between the two variables.

A description of the results obtained is as follows. Over the course of the full 6-month eDNA preservation period, the average SQ value (copies per reaction±SD) was slightly higher for the self-preserved filters (329+72) than it was with the ethanol preserved samples (288+56) (F1,38=4.050, p=0.051; FIG. 3). We did not detect any effect of DNA extraction time (days since collection) on SQ values (F1,38=0.008, p=0.930) or evidence of an interaction between preservation method and extraction time point (F1,38=1.149, p=0.290).

The above experiments infer that there is no significant difference in the environmental DNA preservation capabilities of the self-preserving filter cartridges and the industry-standard ethanol preservation method. Surprisingly, the average SQ values from self-preserved filters were actually slightly higher than those from ethanol-preserved filters. We also did not detect any difference in template environmental DNA quantify on the replicate filters over the course of a six-month preservation trial involving storage at room temperature. This suggests that both methods are effective options for field preservation of environmental DNA captured on filter samples, and that samples of both preservation types can be stored at low cost for up to two months.

Now referring to FIG. 6, which illustrates average environmental DNA quantity (SQ) from replicate filter samples extracted at seven time points after filtration (11 days, 18 days, 25 days, 32 days, 60 days, 88 days, 172 days), and from two preservation treatments: ethanol (black), self-preserving (grey). The experiment compares target environmental DNA degradation between methods over the course of the preservation trial.

Further embodiments include modifications and variations regarding the filter components. Returning to FIGS. 3-5, one could envision that the desiccating elements 314 shows in FIG. 5A could reside on an element separate from the bottom of the housing 310. The housing may still comprise the top 320 and the bottom 310, but the desiccating element 314 would reside inside the housing, rather than the desiccating element being part of the housing.

This has numerous advantages. By moving the desiccating element inside the housing, when the housing is closed, it becomes impervious to any more moisture or contamination. This may also eliminate the need for any secondary storage, such as the bag as used above. The inlet port and outlet port could be capped or otherwise closed off, and the user would only have to store the self-preserving filter cartridge itself.

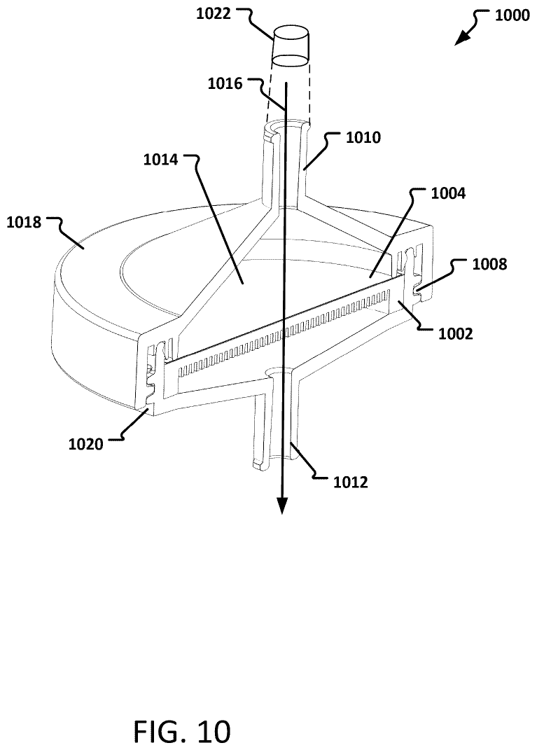

FIGS. 10-14 show different embodiments of filter cartridge assembly cartridges, referred to here as filter assemblies for brevity. Any of the previous components such as connections to external apparatus, etc., will not be repeated here. However, any of those elements may be combined with the embodiments of FIGS. 10-14, without having to repeat those elements here. The embodiments in FIGS. 10-14 have a desiccating element internal to the housing, which forms an interior cavity.

FIG. 10 shows an alternative embodiment of a filter assembly 1000. The filter assembly has screen 1002 and the filter membrane 1004 contained within the interior cavity 1014. One should note that the interior cavity 1014 extends from the top of housing 1018 to the bottom of housing 1020, and includes the desiccating screen and filter, and the space above and below them. In this embodiment, the housing has two pieces, but one could manufacture the filter assembly as one piece that can later be peeled or otherwise opened to access the filter and screen. A groove on the bottom of the housing 1018 fits allows the top and bottom to be screwed together forming the connection 1008. The top and bottom portions of the housing could connect together by a connection that is snapped, threaded, pressure, tear away, and an interference fit with an annular bead. These are just examples and not intended to be exhaustive.

The housing bottom 1020 includes the outlet port 1012, and the housing top 1018 includes the inlet port 1010. The inlet port and the outlet port are fluidically coupled to form a flow path, shown by arrow 1016. The inlet port and the outlet port also comprise connectors designed to allow the fluid assembly to connect to an apparatus, or two. Each connector may comprise a luer connector, a barbed connector, a push connector, threaded pipe fitting, a sanitary tube fitting, or a tapered connector, as examples without limitation. The connectors may also be a combination, wherein the inlet port has one type of connector, and the outlet port has another.

In use, an external apparatus would connect to either the inlet port, the outlet port or both. As water flows along the flow path, the filter membrane has a porosity selected to capture one or more biological molecules such as, but not limited to, nucleic acids, proteins, microbes, animal cells, plant cells, particulates, and combinations thereof. As previously mentioned, the filter membrane may have a backer between the filter membrane and the desiccating screen.

After use, the user can disconnect the filter assembly and cap the ends, an shown at 1022. This eliminates the need for a secondary storage container, such as an envelope, to contain the assembly away from contamination and any further moisture. The housing forms the interior cavity such that the interior cavity is impermeable to liquids and gases. However, the user can also use an impermeable envelope as discussed above.

The desiccating element removes water from the filter membrane while the filter assembly is no longer in the flow path and either the ports are capped or otherwise sealed away from other liquids and gases. This eliminates the need to use a desiccating gas to remove water from the membrane. However, if desired, the user could add an additional desiccant to the interior cavity. This will be discussed in more detail in further embodiments where it is easier to show the desiccant but would apply to all embodiments.

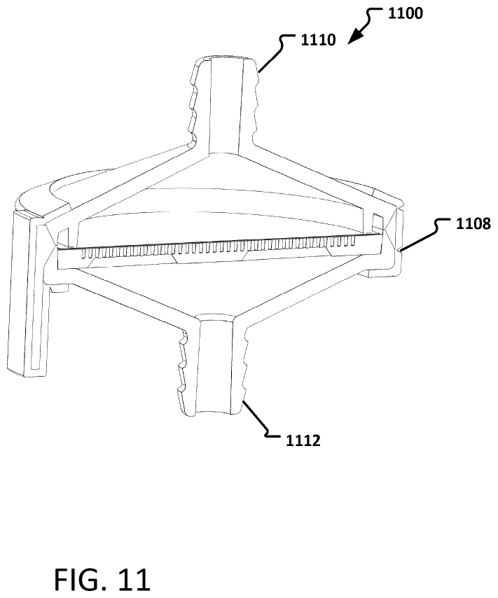

Having seen all of the elements of a housing that forms an interior cavity, other embodiments show different variations. FIG. 11 shows a filter assembly 1100 with most of the same components and features as shown in FIG. 11. In this embodiment, the connectors to the external apparatus on the inlet port 1110, and outlet port 1112 comprise barbed connectors. The connection between the top and bottom of the housing comprises a tear away connection, shown by the resulting groove 1108 that would remain after the portion is torn off the housing.

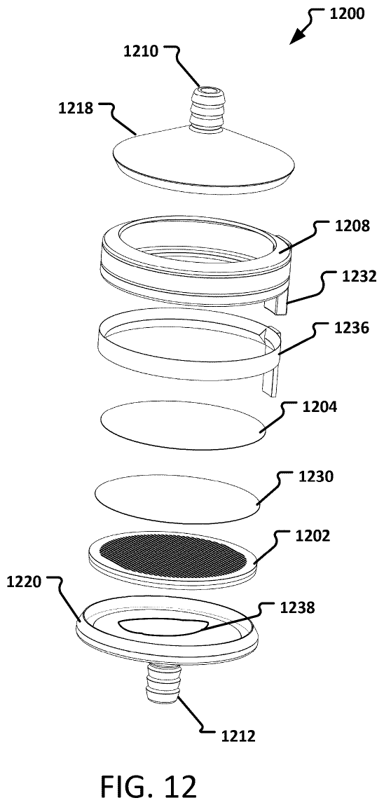

FIG. 12 shows another alternative embodiment of a filter assembly 1200. In this embodiment, the top 1218 has an annular ring connector on the inlet port 1210. The connection between the top 1218 and the bottom 1220 comprises an over molded portion on the top 1208. A spine 1236 fits inside the top 1208, with a tab 1238 that fits with the tab 1232 on the top 1208. After use, when the user wants to access the filter membrane 1204, the user would pull the tab, and it would peel the ring of the connection 1208 to open the interior cavity. Also shown here is the optional filter backer 1230. Again, components shown in one embodiment may apply to other embodiments. The filter backer 1230 controls the porosity of the filter membrane 1204, so it may not be needed. FIG. 12 also shows an additional desiccant material 1238 on the inside of the interior cavity, in addition to the desiccating screen 1212.

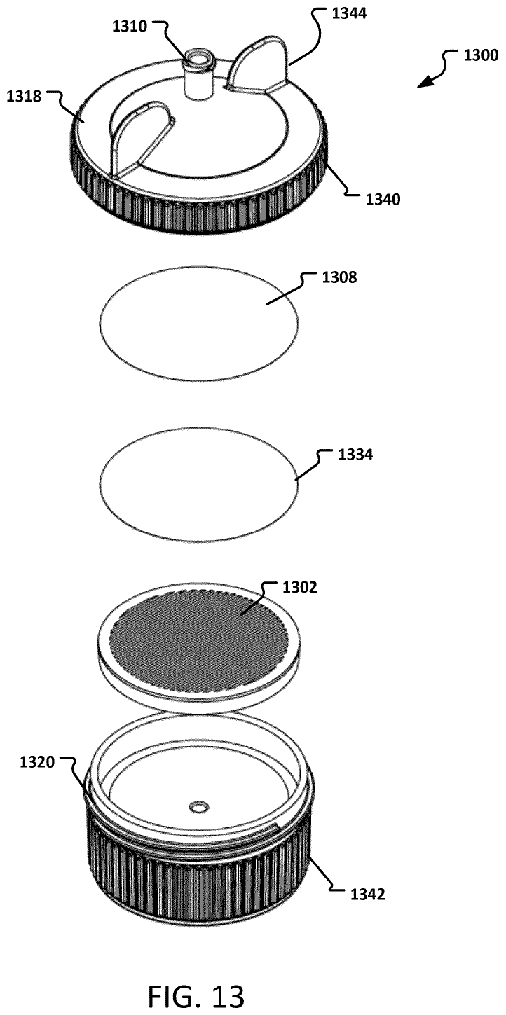

FIG. 13 shows an embodiment of a filter assembly 1300 that includes the filter backer 1334. In FIG. 13, the top 1318 holds the inlet port 1310 and further includes thumb screws such as 1344. The bottom 1320 would include those as well but are not seen in this view. The top 1318 and the bottom 1320 have textured sides, or skirts, 1340 and 1342 respectively, to allow the user to grasp the assembly and twist the top and the bottom apart or together. The interior cavity formed by the top and bottom would contain the filter 1308, the filter backer 1334, and the desiccating screen 1302. As mentioned in previous embodiments, the bottom would contain the outlet port not shown in this view.

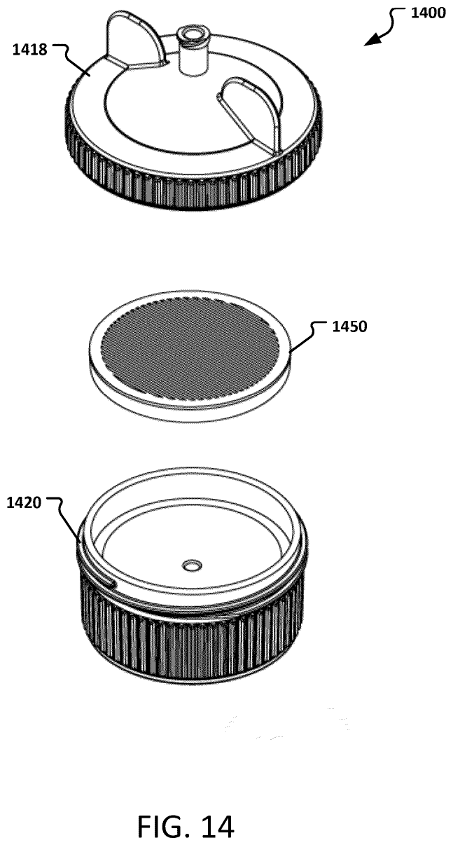

FIG. 14 shows an embodiment of the filter assembly 1400 that does not contain a filter backer. This embodiment has a combined screen and filter 1450. This screen and filter material would be combined into one element 1450 of the assembly, contained within the interior cavity formed by the top 1418 and the bottom 1420.

In this manner, one can provide an internally desiccated filter assembly that does not require a secondary impermeable storage, or the use of a desiccating gas, to desiccate the filter. These embodiments have several advantages. The advantages include not needing the secondary impermeable storage, the housing forms an interior cavity that is impermeable to gases and liquids, can be used at ambient temperatures, and has inlet ports and outlet ports that can be capped to seal off the interior cavity.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Although specific aspects of this disclosure have been illustrated and described for purposes of illustration, it will be understood that various modifications may be made without departing from the spirit and scope of the invention. Accordingly, the invention should not be limited except as by the appended claims.

1. A filter assembly, comprising:
a housing having an inlet port, an outlet port, and an interior compartment, the inlet port, and the outlet port fluidically coupled to form a flow path in the interior compartment;
a filter membrane in the flow path, the filter membrane having a porosity capable of capturing biological molecules as liquid flows along the flow path;
a fluid screen in the flow path on an opposite side of the filter membrane from the inlet port; and
a desiccating element in the flow path comprised of one of a hygroscopic or hydrophilic material selected to absorb moisture from the filter membrane at ambient temperature.

2. The filter assembly as claimed in claim 1, wherein the housing comprises a first part containing the inlet port, and a second part containing the outlet port, the second part being connected to the first part to form the interior compartment.

3. The filter assembly as claimed in claim 2, wherein the first and second parts are connected by one of a connection selected from the group consisting of: threaded, pressure, tear away, and interference fit with an annular bead.

4. The filter assembly as claimed in claim 1, wherein the desiccating element is one of either part of the fluid screen, or a separate component from any other component in the interior compartment.

5. The filter assembly as claimed in claim 1, wherein the filter membrane has a porosity to capture one or more biological molecules selected from the group consisting of: nucleic acids, proteins, microbes, animal cells, plant cells, particulates, and combinations thereof.

6. The filter assembly as claimed in claim 1, wherein the outlet port and the inner port are configured to connect to a fluid filtering apparatus by one selected from the group consisting of: a luer connector, a barbed connector, push connector, threaded pipe fitting, a sanitary tube fitting, a tapered connector, or combinations thereof.

7. The filter assembly as claimed in claim 1, wherein the desiccating element lies next to the filter membrane on a side of the filter membrane opposite the inlet port.

8. The filter assembly as claimed in claim 1, wherein the housing makes the interior compartment impermeable to liquids and gases.

9. The filter assembly as claimed in claim 1, further comprising a filter backer in the flow path next to the filter membrane.

10. The filter assembly as claimed in claim 1, further comprising caps that fit over the inlet port and the outlet port to seal the interior compartment.

11. The filter assembly as claimed in claim 1, further comprising an impermeable envelope to contain the filter assembly after removal from a fluid filtering apparatus.

12. The filter assembly as claimed in claim 1, further comprising an additional desiccant inside the interior compartment.

13. A filter assembly, comprising:
a housing having an inlet port, an outlet port, and an interior compartment, the inlet port, and the outlet port fluidically coupled to form a flow path in the interior compartment;
a filter membrane in the flow path, the filter membrane having a porosity capable of capturing biological molecules as liquid flows along the flow path;
a fluid screen in the flow path on an opposite side of the filter membrane from the inlet port; and
a desiccating element in the flow path comprised of one of a hygroscopic or hydrophilic material selected to absorb moisture from the filter membrane at ambient temperature without use of a desiccating gas.

14. A filter assembly, comprising:
a housing having an inlet port, an outlet port, and an interior compartment, the inlet port, and the outlet port fluidically coupled to form a flow path in the interior compartment;
a filter membrane in the flow path, the filter membrane having a porosity capable of capturing biological molecules as liquid flows along the flow path;
a fluid screen in the flow path on an opposite side of the filter membrane from the inlet port; and
a desiccating element in the flow path comprised of one of a hygroscopic or hydrophilic material selected to absorb moisture from the filter membrane at ambient temperature without use of a secondary impermeable storage.
